(19) United States
(12) Patent Application Publication
(10) Pub. No.: US 2025/0069304 A1
(43) Pub. Date: Feb. 27, 2025

(54) Data Processing Method, Attenuation Coefficient Image Generation Method, and Image Generation Method

(71) Applicant: SHIMADZU CORPORATION, Kyoto-shi (JP)

(72) Inventor: Yui SHIGEKI, Kyoto-shi (JP)

(21) Appl. No.: 18/809,770

(22) Filed: Aug. 20, 2024

(30) Foreign Application Priority Data
Aug. 22, 2023 (JP) 2023-134496

(51) Int. Cl. G06T 11/60; G06T 5/20; G06T 7/50; G06T 7/70; G06V 10/75

(52) U.S. Cl. CPC G06T 11/60; G06T 5/20; G06T 7/50; G06T 7/70; G06V 10/758; G06T 2207/10104; G06T 2207/10108

(57) ABSTRACT

A data processing method for generation of a test image from a target image which is a target of estimation with a machine learning model is provided. The test image is inputted to the machine learning model for estimation. The machine learning model has been subjected to machine learning processing with a pair of a basic image and an output image being defined as training data. The data processing method generates the test image by performing on the target image, adjustment processing based on a feature of the basic image.

### CROSS REFERENCE TO RELATED APPLICATIONS

This nonprovisional application is based on Japanese Patent Application No. 2023-134496 filed with the Japan Patent Office on Aug. 22, 2023, the entire contents of which are hereby incorporated by reference.

### BACKGROUND OF THE INVENTION

### Field of the Invention

The present invention relates to data processing on a test image inputted to a machine learning model.

### Description of the Background Art

In an example where test data is applied to a machine learning model trained with training data to obtain a result of estimation, when there is a qualitative difference called a domain gap between an environment where training data is collected and an environment where test data is collected, the machine learning model is basically unable to exhibit expected performance for the test data.

In an approach to reduction of the domain gap, for example, transfer learning and domain adaptation are available. In transfer learning, additional learning (fine tuning) using data (data with ground truth) collected in a test environment is performed on a machine learning model trained with data collected in a training environment.

Domain adaptation is a learning method with the use of both of data collected in a training environment and data collected in a test environment. Specific examples of approaches adopted in domain adaptation include an approach to use of a pseudo label, an approach to use of a generation model, and an approach to conversion of a domain of training data into a domain of the test environment. This method does not require data in the test environment to be given a ground truth label in advance.

As described, for example, in Asymmetric Tri-training for Unsupervised Domain Adaptation, [URL: https://arxiv.org/pdf/1702.08400.pdf], in an approach to use of a pseudo label, initially, data in a test environment is tested with a machine learning model trained with training data, and with a result of the test being defined as pseudo ground truth, machine learning processing using both of training data and data collected in the test environment is performed.

For example, as described in Adapting Visual Category Models to New Domains (K. Saenko et al. 2010), [URL:
https://people.bu.edu/bkulis/pubs/saenko_eccv_2010.pdf] or Deep Domain Confusion: Maximizing for Domain Invariance (E. Tzeng et al. 2014), [URL:
https://arxiv.org/pdf/1412.3474.pdf], in an approach to use of a generation model, both of training data and data collected in a test environment are used to perform machine learning processing with such a feature extractor that distributions of feature values obtained from the training data and the data collected in the test environment match with each other, regardless of a domain.

As described in FDA: Fourier Domain Adaptation for Semantic Segmentation (Y. Yang et al. 2020, [URL: arxiv.org/pdf/2004.05498.pdf], in an approach to conversion of a domain of training data to a domain of data in a test environment, domain information of the test environment is used to convert the domain of the training data to the domain of the test environment and machine learning processing using the converted training data is performed.

### SUMMARY OF THE INVENTION

Preparation of a large amount of data collected in a test environment is required for both of transfer learning and domain adaptation described above. Furthermore, when there are a plurality of test environments, preparation of data is required for each test environment. Basically, both of transfer learning and domain adaptation described above require works which are learning requiring trial and error.

The present invention was made in view of such circumstances, and an object thereof is to provide a technique to suppress burdens imposed on a user for reduction of a domain gap.

A data processing method according to one aspect of the present disclosure is a data processing method for generation of a test image, the data processing method being to be performed by a computer and applied to a machine learning model subjected to machine learning processing using a basic image. The data processing method includes obtaining, by circuitry of the computer, a target image in a domain different from a domain where the basic image is obtained and generating, by the circuitry of the computer, the test image by performing on the target image, adjustment processing based on a feature corresponding to the domain where the basic image is obtained.

An attenuation coefficient image generation method according to one aspect of the present disclosure is to be performed by a computer and includes generating, by circuitry of the computer, an intermediate image including an image relating to a tissue area based on a test image generated according to the data processing method described above and generating, by the circuitry of the computer, an attenuation coefficient image based on the intermediate image and a known attenuation coefficient of the tissue area.

An image generation method according to one aspect of the present disclosure is an image generation method using a machine learning model. The image generation method is to be performed by a computer. The machine learning model has been subjected to machine learning processing using basic data. The image generation method includes obtaining, by circuitry of the computer, a target image obtained in a domain different from a domain where the basic data is obtained, generating, by the circuitry of the computer, a test image by performing on the target image, adjustment processing based on a feature corresponding to the domain where the basic data is obtained, and obtaining an output image by applying the test image to the machine learning model.

The foregoing and other objects, features, aspects and advantages of this invention will become more apparent from the following detailed description of this invention when taken in conjunction with the accompanying drawings.

### BRIEF DESCRIPTION OF THE DRAWINGS

FIG. 1 is a diagram showing a configuration of a positron emission tomography (PET) apparatus 1.

FIG. 2 is a diagram showing a configuration of a radiation detector 3.

FIG. 3 is a flowchart of radioactivity distribution image generation processing.

FIG. 4 is a diagram schematically showing a flow of radioactivity distribution image generation processing.

FIG. 5 is a diagram for illustrating exemplary generation of an intermediate image using a machine learning model.

FIG. 6 is a diagram for illustrating exemplary generation of an attenuation coefficient image through conversion processing.

FIG. 7 is a diagram for illustrating exemplary training of a machine learning model 8.

FIG. 8 is a diagram for illustrating exemplary adjustment processing.

FIG. 9 is a diagram for illustrating other exemplary adjustment processing.

FIGS. 10 to 12 are diagrams for still other exemplary adjustment processing.

FIG. 13 is a diagram for more specifically illustrating adjustment processing using a pre-processing mask 640.

### DESCRIPTION OF THE PREFERRED EMBODIMENTS

An embodiment of the present disclosure will be described in detail below with reference to the drawings. The same or corresponding elements in the drawings have the same reference characters allotted and description thereof will not be repeated.

In the present disclosure, an image subjected to adjustment processing is used as a test image to be inputted to a machine learning model. In the present disclosure, a target image generated from measurement data from a PET apparatus is adopted as a mere example of an image to be used for generation of a test image. The adjustment processing according to the present disclosure may be applicable not only to an image generated from measurement data from the PET apparatus but also to any type of image to be inputted to the machine learning model (for example, a computed tomography (CT) image, a magnetic resonance imaging (MRI) image, a radiographic image, an ultrasonic image, a microscopic image, and a mass spectrographic image).

### [Configuration of PET Apparatus]

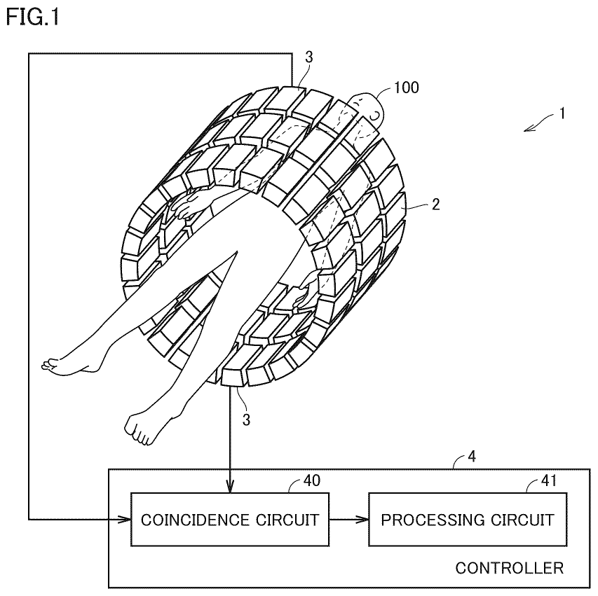

FIG. 1 is a diagram showing a configuration of a PET apparatus 1. As shown in FIG. 1, PET apparatus 1 is an apparatus for imaging of a subject 100 by detection of radioactive rays (gamma rays) generated from the inside of subject 100, the radioactive rays originating from a radiopharmaceutical agent administered in advance to subject 100. Subject 100 is, for example, a human. Radioactive rays (gamma rays) are annihilation radioactive rays generated by annihilation between positrons generated from the radiopharmaceutical agent and electrons of atoms in the vicinity of positrons in the inside of subject 100.

PET apparatus 1 is configured to generate a radioactivity distribution image of subject 100 based on a result of imaging of subject 100. PET apparatus 1 may image the whole body of subject 100 or a part (breast, head, and the like) of subject 100.

PET apparatus 1 includes a detector ring 2 that surrounds subject 100. Detector ring 2 is provided in such a manner that a plurality of detector rings are stacked in a direction of a body axis of subject 100. In the inside of detector ring 2, a plurality of radiation (gamma ray) detectors 3 are provided. Detector ring 2 is thus configured to detect radioactive rays (gamma rays) generated from the radiopharmaceutical agent in subject 100.

PET apparatus 1 includes a controller 4. In one implementation, controller 4 is implemented by a general-purpose information processing apparatus including a processor and a memory where a given program and/or data is/are stored in a non-transitory manner. Controller 4 includes a coincidence circuit 40 and a processing circuit 41. Coincidence circuit 40 and processing circuit 41 may each be implemented as dedicated hardware circuitry or by execution of a given program by the processor. Controller 4 may be implemented by distributed processing by a plurality of computers. At least one of the plurality of computers may include a computer arranged at a location remote from PET apparatus 1. The program may be distributed as a program product containing the program.

Though FIG. 1 shows only wiring from two radiation detectors 3 to controller 4 (coincidence circuit 40), controller 4 is actually connected to each of the plurality of radiation detectors 3.

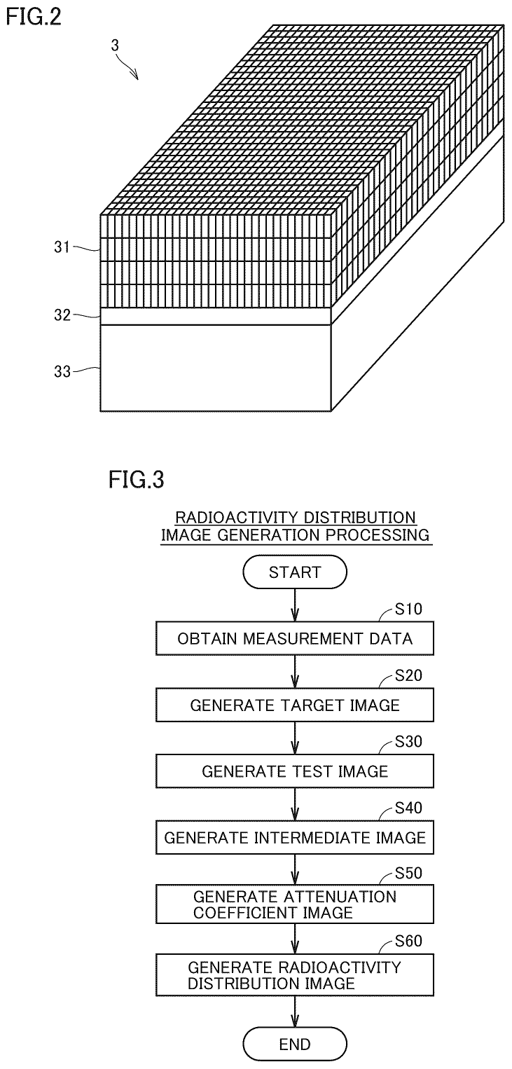

FIG. 2 is a diagram showing a configuration of radiation detector 3. The configuration of PET apparatus 1 will be described in further detail further with reference to FIG. 2.

As shown in FIG. 2, radiation detector 3 includes a scintillator block 31, a light guide 32, and a photo multiplier tube (PMT) 33. Light guide 32 may not be provided in radiation detector 3. For example, a sensor such as a silicon photomultiplier (SiPM) other than the PMT may be employed in radiation detector 3.

Scintillator block 31 converts radioactive rays (gamma rays) from subject 100 to which the radiopharmaceutical agent is administered into light. As the radiopharmaceutical agent is administered to subject 100, positrons of positron emission type RI extinct and two radioactive rays (gamma rays) are generated. Each scintillator element included in scintillator block 31 converts radioactive rays (gamma rays) from subject 100 into light.

Light guide 32 is optically coupled to each of scintillator block 31 and photo multiplier tube 33. In scintillator block 31, light generated in the scintillator element is diffused, and diffused light is inputted to photo multiplier tube 33 through light guide 32.

Photo multiplier tube 33 multiplies light inputted through light guide 32 to convert light into an electrical signal. This electrical signal is transmitted to coincidence circuit 40.

Coincidence circuit 40 generates detection signal data (count value) based on the electrical signal transmitted from photo multiplier tube 33.

More specifically, coincidence circuit 40 checks a position of scintillator block 31 and timing of incidence of radioactive rays (gamma rays), and determines a transmitted electrical signal as proper data only when radioactive rays (gamma rays) are simultaneously incident on two scintillator blocks 31 located on opposing sides of subject 100 (on a diagonal line with subject 100 being defined as the center). In other words, coincidence circuit 40 detects based on the electrical signal described above, simultaneous observation (that is, coincidence) of radioactive rays (gamma rays) in two radiation detectors 3 located on opposing sides of subject 100 (on the diagonal line with subject 100 being defined as the center).

The detection signal data (count value) composed of proper data determined as coincidence by coincidence circuit 40 is transmitted to processing circuit 41.

Processing circuit 41 generates a radioactivity distribution image of the inside of subject 100 based on detection of radioactive rays (gamma rays) by detector ring 2.

### [Generation of Radioactivity Distribution Image]

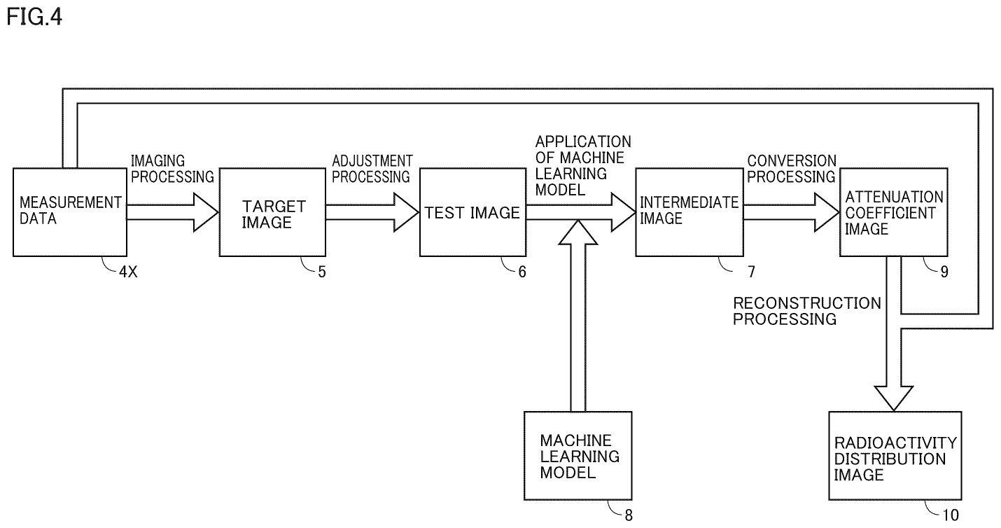

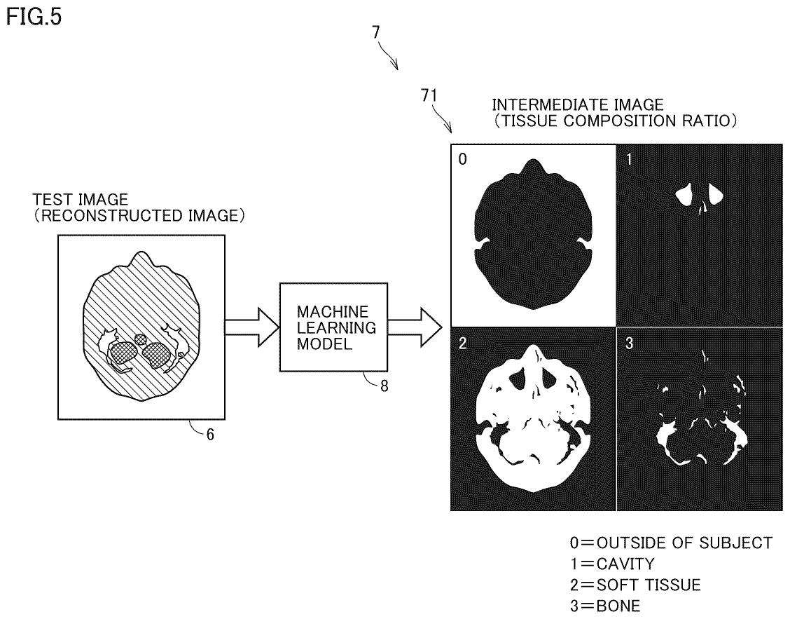

Radioactivity distribution image generation processing by PET apparatus 1 will now be described with reference to FIG. 3. FIG. 3 is a flowchart of the radioactivity distribution image generation processing. In the description of the radioactivity distribution image generation processing, FIG. 4 is also referred to. FIG. 4 is a diagram schematically showing a flow of the radioactivity distribution image generation processing.

The radioactivity distribution image generation processing is performed by processing circuit 41 of controller 4. In one implementation, the radioactivity distribution image generation processing is started in response to an operation performed to start the processing in PET apparatus 1.

In step S10, processing circuit 41 obtains measurement data (“measurement data 4X” in FIG. 4) from radiation detector 3 based on detection of radioactive rays emitted from subject 100.

In step S20, processing circuit 41 generates a target image (a “target image 5” in FIG. 4) by imaging processing on the measurement data.

The target image may include at least one of a three-dimensional image, an axial cross-sectional image, a coronal cross-sectional image, a sagittal cross-sectional image, a patch image obtained by extraction of a partial area from the three-dimensional image, a patch image obtained by extraction of a partial area from the axial cross-sectional image, a patch image obtained by extraction of a partial area from the coronal cross-sectional image, and a patch image obtained by extraction of a partial area from the sagittal cross-sectional image.

The “cross-sectional image” means a two-dimensional image of one slice. The “axial cross-sectional image” means an image of a cross-section orthogonal to the body axis. The “coronal cross-sectional image” means an image of a cross-section along a coronal plane in parallel to the body axis. The “sagittal cross-sectional image” means an image of a cross-section along a sagittal plane in parallel to the body axis. The target image may be an image only of one slice or a set of images of successive several slices.

Examples of imaging processing in step S20 include imaging processing by conversion into histograms, imaging processing by machine learning, or processing including back projection processing. For the imaging processing by conversion into histograms, a method of imaging by addition of an event to a position highest in probability based on time of flight (TOF) information included in measurement data may be adopted. For the imaging processing by machine learning, a method of imaging with a machine learning model for conversion of measurement data into the target image can be adopted. For the processing including back projection processing, for example, simple back projection processing and reconstruction processing can be adopted.

In step S20, the target image may be generated without at least one of attenuation correction processing and scatter correction processing. The attenuation correction processing refers to processing for correction of attenuation of radioactive rays in the inside of subject 100. The scatter correction processing refers to processing for correction of scatter of radioactive rays in the inside of subject 100. In step S20, a target image without being corrected, which was not subjected to at least one of the attenuation correction processing and the scatter correction processing, may be generated from the measurement data.

In step S20, image quality conversion processing does not have to be performed, image quality conversion processing may be performed, or area identification processing may be performed. In the present embodiment, the target image may include at least one of an image to which the image quality conversion processing was not applied, an image to which the image quality conversion processing was applied, and an image to which the area identification processing was applied.

For the image quality conversion processing, for example, y correction processing, histogram equalization processing, smoothing processing, edge detection processing, and the like can be adopted. For the image quality conversion processing, processing for adding random noise of a distribution such as uniform distribution, normal distribution, Poisson distribution, and Laplacian distribution may be adopted. For the image quality conversion processing, processing for multiplying the entire image or a specific area in an image by a constant may be adopted. For the area identification processing, processing for identification of a contour of subject 100 in an image may be adopted.

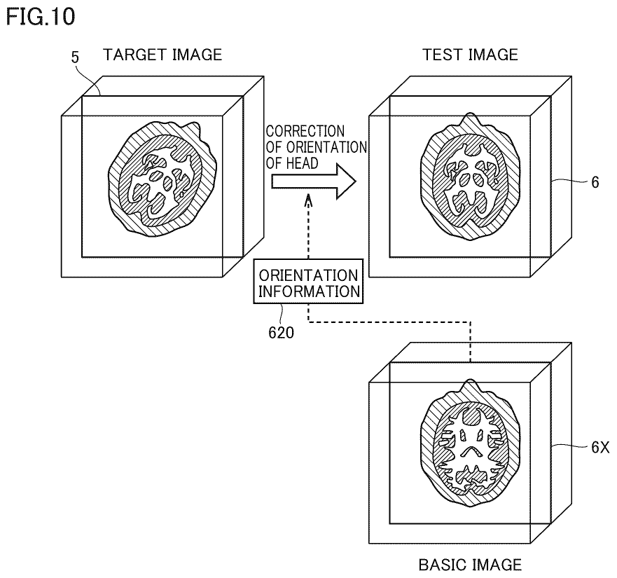

In step S30, processing circuit 41 performs adjustment processing on the target image to generate a test image (a “test image 6” in FIG. 4). The adjustment processing on the target image will be described later with reference to FIGS. 8 to 13.

In step S40, processing circuit 41 applies the test image to a machine learning model (a “machine learning model 8” in FIG. 4) for an intermediate image to generate the intermediate image (an “intermediate image 7” in FIG. 4). The intermediate image is an exemplary “result of estimation” by the machine learning model.

A known model (for example, “machine learning model 8” described in WO2021/260928) may be adopted as machine learning model 8.

Machine learning model 8 is a machine learning model that receives test image 6 as input and provides intermediate image 7 as output. Machine learning model 8 includes at least one of a machine learning model that receives a three-dimensional image as input, a machine learning model that receives an axial cross-sectional image as input, a machine learning model that receives a coronal cross-sectional image as input, a machine learning model that receives a sagittal cross-sectional image as input, a machine learning model that receives a patch image extracted from the three-dimensional image as input, a machine learning model that receives a patch image extracted from the axial cross-sectional image as input, a machine learning model that receives a patch image extracted from the coronal cross-sectional image as input, and a machine learning model that receives a patch image extracted from the sagittal cross-sectional image as input.

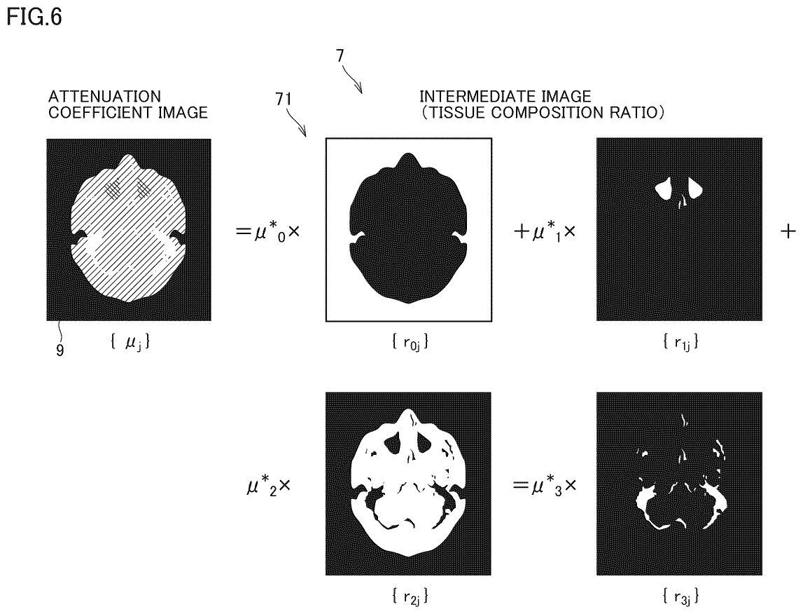

FIG. 5 is a diagram for illustrating exemplary generation of an intermediate image using the machine learning model. FIG. 5 shows an example in which the machine learning model outputs the intermediate image corresponding to the axial cross-sectional image in response to input of the axial cross-sectional image as the test image, for the sake of convenience of description.

Intermediate image 7 is composed of combination of N (a finite number of) tissues an attenuation coefficient of which has been known, such as brain, bone, skin, muscles, and internal organs. For example, when measurement data 4X falls under measurement data of the head of a human, an element (tissue) included in an image relating to a tissue area of intermediate image 7 includes at least one of a background (outside of the subject), a cavity (a nasal cavity, a mouth cavity, and the like), a soft tissue (brain, skin, and the like), and bone (skull). For example, when measurement data 4X falls under measurement data of breast of a human, an element (tissue) included in an image relating to a tissue area of intermediate image 7 includes at least one of the background (outside of the subject) and the soft tissue.

As shown in FIG. 5, intermediate image 7 includes a tissue composition ratio image 71 indicating a ratio of tissues included in each pixel as an image relating to the tissue area. Tissue composition ratio image 71 is a multi-channel image having a ratio of a plurality of tissues included in each pixel as a pixel value. In the example shown in FIG. 5, tissue composition ratio image 71 is an image of the head of the human, and includes images of four channels which are a background image channel, a cavity image channel, a soft tissue image channel, and a bone image channel. The image of a background channel is configured such that the ratio of the background included in each pixel is shown as a pixel value. The image of a cavity channel is configured such that the ratio of the cavity included in each pixel is shown as a pixel value. The image of a soft tissue channel is configured such that the ratio of the soft tissue included in each pixel is shown as a pixel value. The image of a bone channel is configured such that the ratio of the bone included in each pixel is shown as a pixel value. Since the pixel value of each of the images of the four channels indicates the ratio, the sum of the pixel values of the images of the four channels for each pixel is 1.

Referring back to FIGS. 3 and 4, in step S50, processing circuit 41 performs conversion processing on the intermediate image to generate an attenuation coefficient image (an “attenuation coefficient image 9” in FIG. 4).

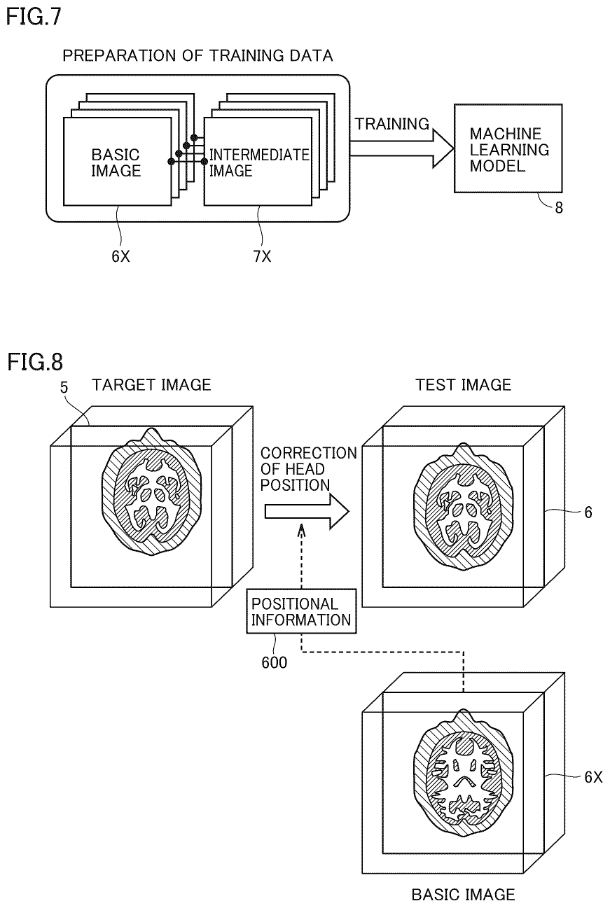

FIG. 6 is a diagram for illustrating exemplary generation of an attenuation coefficient image through the conversion processing. Exemplary conversion processing is linear combination of tissue composition ratio image 71 using a known attenuation coefficient. In this example, each tissue in tissue composition ratio image 71 is combined with the attenuation coefficient of each tissue to generate attenuation coefficient image 9. More specifically, linear combination processing of tissue composition ratio image 71 of each tissue with the known attenuation coefficient being defined as the coefficient is performed, in accordance with an expression (1) below.

$${μ}_{j} = {\sum }_{n = 1}^{N} {μ}_{n}^{*} {r}_{nj} \qquad ( 1 )$$

n: tissue label (tissue number)
j: pixel number
μⱼ: attenuation coefficient of pixel j
μ*ₙ: attenuation coefficient (known attenuation coefficient) of tissue n
rₙⱼ: composition ratio of tissue n of pixel j

The “composition ratio of tissue n of pixel j” used in the expression (1) satisfies an expression (2) below.

$$0 \leq {r}_{nj} \leq 1 , \qquad ( 2 )$$

$${\sum }_{n = 1}^{N} {r}_{nj} = 1$$

For example, when tissue composition ratio image 71 is the image of the head of the human and includes the images of the four channels of the background, the cavity, the soft tissue, and the bone, processing circuit 41 uses an attenuation coefficient μ*₀ of the background, an attenuation coefficient μ*₁ of the cavity, an attenuation coefficient μ*₂ of the soft tissue, and an attenuation coefficient μ*₃ of the bone which are generally known, to perform the linear combination processing of tissue composition ratio image 71 of each tissue, with the known attenuation coefficient being defined as the coefficient, in accordance with the expression (1) above.

Referring back to FIGS. 3 and 4, in step S60, processing circuit 41 generates a radioactivity distribution image (a “radioactivity distribution image 10” in FIG. 4) through reconstruction processing based on attenuation coefficient image 9 and measurement data 4X. In the reconstruction processing, at least one of the attenuation correction processing and the scatter correction processing is performed based on attenuation coefficient image 9. In one implementation, in step S60, the attenuation correction processing is performed based on attenuation coefficient image 9, and the scatter correction processing is performed based on scatter distribution data obtained based on attenuation coefficient image 9 and measurement data 4X. In step S60, quantitative radioactivity distribution image 10 is generated through the attenuation correction processing and the scatter correction processing.

To be strict, attenuation coefficient image 9 used in the reconstruction processing is an image obtained by performing processing for canceling contents in the adjustment processing on the attenuation coefficient image generated in the conversion processing on intermediate image 7.

For example, the contents in the adjustment processing are assumed as clockwise rotation by 100 of target image 5. In this case, attenuation coefficient image 9 used in the reconstruction processing is an image obtained by counterclockwise rotation by 10° of the attenuation coefficient image generated in the conversion processing on intermediate image 7.

The contents in the adjustment processing are assumed as movement of target image 5 by five pixels in a first direction. In this case, attenuation coefficient image 9 used in the reconstruction processing is an image obtained by movement of the attenuation coefficient image generated in the conversion processing on intermediate image 7 by five pixels in a second direction (a direction indicating an orientation opposite to the first direction).

For the reconstruction processing, for example, analytical reconstruction processing and/or iterative reconstruction processing may be adopted. For the analytical reconstruction processing, for example, filtered back projection (FBP) may be adopted. For the iterative reconstruction processing, for example, ordered subsets expectation maximization (OSEM) may be adopted.

Thereafter, processing circuit 41 quits the process in FIG. 3.

### [Machine Learning Model for Intermediate Image]

Machine learning model 8 for the intermediate image to be used in PET apparatus 1 will now be described.

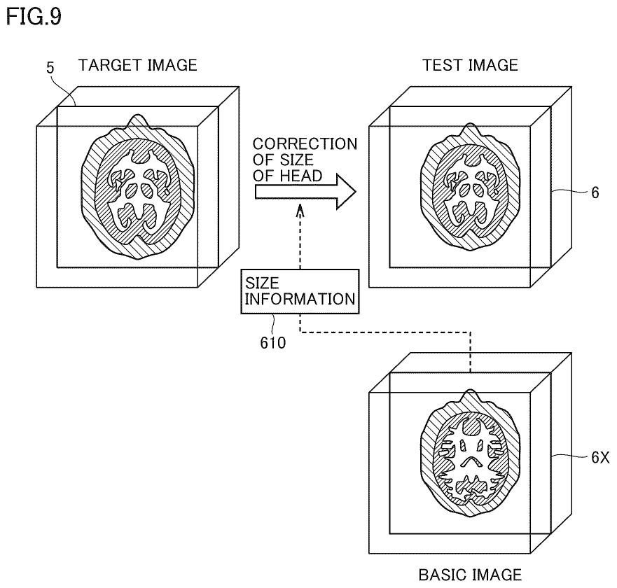

FIG. 7 is a diagram for illustrating exemplary training of machine learning model 8. FIG. 7 shows a pair of a basic image 6X and an intermediate image 7X as training data. Machine learning model 8 is generated by machine learning processing on a training model. Exemplary machine learning processing to be performed is supervised learning where a plurality of pairs of basic image 6X and intermediate image 7X are used as training data as shown in FIG. 7. In one implementation, the machine learning processing is performed by processing circuit 41 of controller 4.

Each of a plurality of basic images 6X is an image generated by imaging processing on measurement data from radiation detector 3 and corresponding to “target image 5” described above. Each of a plurality of intermediate images 7X is an image corresponding to “intermediate image 7” described above.

Machine learning model 8 includes a deep neural network. The deep neural network of machine learning model 8 includes convolutional processing. In other words, machine learning model 8 includes a deep convolutional neural network. For example, a U-shaped network (U-Net) including skip connection can be adopted as the deep convolutional neural network of machine learning model 8. A Softmax function can be adopted as an activation function for the deep convolutional neural network of machine learning model 8.

### [Adjustment Processing on Target Image]

As described with reference to FIG. 3, pre-processing referred to as the adjustment processing is performed on an image to be applied to machine learning model 8. Specifically, as the adjustment processing is performed on target image 5 generated as a target of estimation by machine learning model 8 (generation of the intermediate image with the use of machine learning model 8), test image 6 to be inputted to machine learning model 8 is generated. In the adjustment processing, a feature of basic image 6X used for generation of machine learning model 8 is used. A specific example of the adjustment processing will be described below.

### <Adjustment Processing (1)>

FIG. 8 is a diagram for illustrating exemplary adjustment processing.

In this example, a position of the head in basic image 6X is adopted as a feature of basic image 6X. More specifically, positional information 600 is generated as the feature of basic image 6X. Positional information 600 indicates a position resulting from statistical processing of positions of the center of gravity of the head in a plurality of basic images 6X. Exemplary statistical processing is derivation of an average position. In an example where an image is defined in an XY plane, an X coordinate of positional information 600 is an average value of X coordinates of N positions of the center of gravity, and a Y coordinate of positional information 600 is an average value of Y coordinates of the N positions of the center of gravity. So long as positional information 600 represents the feature of the position of the head in the plurality of basic images 6X, the statistical processing is not limited to derivation of the average position. The X coordinate of positional information 600 may be a median value of the X coordinates of the N positions of the center of gravity and the Y coordinate of positional information 600 may be a median value of the Y coordinates of the N positions of the center of gravity.

In derivation of the feature, an area of the head in each basic image 6X may be identified by a user or by pattern recognition.

In the adjustment processing in this example, as shown in FIG. 8, processing for translating all pixels of target image 5 is performed to move the position of the center of gravity of target image 5 to the position identified in positional information 600 so that test image 6 is generated.

### <Adjustment Processing (2)>

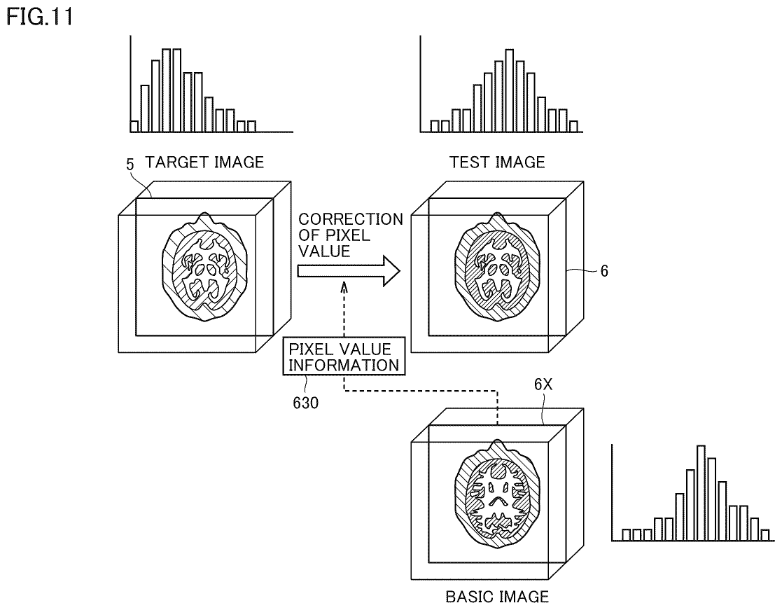

FIG. 9 is a diagram for illustrating other exemplary adjustment processing.

In this example, a shape of the head in basic image 6X is adopted as a feature of basic image 6X. More specifically, size information 610 is generated as the feature of basic image 6X. Size information 610 indicates a value resulting from statistical processing of dimensions in a given direction of the head in a plurality of basic images 6X. Exemplary statistical processing is derivation of an average value. For example, size information 610 is an average value of dimensions in the given direction in the plurality of basic images 6X. The given direction is defined, for example, by an axis that defines a plane such as an X axis or a Y axis in the example where the image is defined in the XY plane. So long as size information 610 represents the feature of the size in the plurality of basic images 6X, the statistical processing may be other types of processing such as derivation of a median value.

In the adjustment processing in this example, as shown in FIG. 9, target image 5 is zoomed up or down such that the dimension in the given direction of the area of the head in target image 5 has a value defined in size information 610, and thus test image 6 is generated.

In an example where both of basic image 6X and target image 5 are a set of images of a plurality of slices, size information 610 may be a statistical value of a volume of the head defined by the images of the plurality of slices of basic image 6X. In this case, in the adjustment processing, each of the images of the plurality of slices of target image 5 is zoomed up or down such that the volume of the head defined by the images of the plurality of slices of target image 5 matches with the statistical value identified by size information 610, and thus the plurality of slices that make up test image 6 are generated.

### <Adjustment Processing (3)>

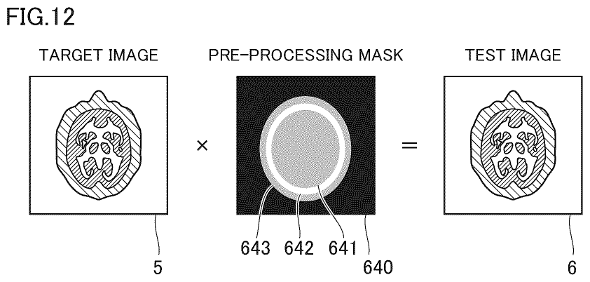

FIG. 10 is a diagram for illustrating still other exemplary adjustment processing.

In this example, a posture of the head in basic image 6X is adopted as a feature of basic image 6X. More specifically, orientation information 620 is generated as the feature of basic image 6X. Orientation information 620 indicates a value resulting from statistical processing of values indicating orientations in a plurality of basic images 6X. Exemplary statistical processing is derivation of an average value. In the example where each basic image 6X is defined in the XY plane, ellipse curve fitting is performed on each basic image 6X. An ellipse corresponding to the head is thus estimated. An average value of inclinations with respect to the X axis or the Y axis of major axes of the ellipses estimated for the plurality of basic images 6X is identified as orientation information 620. The statistical processing is not limited to derivation of the average value so long as orientation information 620 represents the feature of the orientation of the head in the plurality of basic images 6X.

In the adjustment processing in this example, as shown in FIG. 10, ellipse curve fitting is performed on target image 5, the inclination of the major axis of the estimated ellipse is derived, and rotation processing is performed on target image 5 such that the derived inclination is equal to the inclination represented in orientation information 620. Test image 6 is thus generated.

### <Adjustment Processing (4)>

FIG. 11 is a diagram for illustrating still other exemplary adjustment processing.

In this example, contrast in basic image 6X is adopted as a feature of basic image 6X. More specifically, pixel value information 630 is generated as the feature of basic image 6X. Pixel value information 630 defines a statistical value of a frequency for each pixel value in a plurality of basic images 6X. Though an exemplary statistical value is an average value, the statistical value may be a value of another type such as a median value so long as pixel value information 630 represents the feature about contrast in the plurality of basic images 6X.

In the adjustment processing in this example, as shown in FIG. 11, contrast of target image 5 is adjusted such that a distribution of frequencies of the pixel values of target image 5 is equal (or closer) to a distribution of frequencies defined by pixel value information 630, and thus test image 6 is generated.

### <Adjustment Processing (5)>

FIG. 12 is a diagram for illustrating still other exemplary adjustment processing.

In this example, a ratio of a pixel value between an outer periphery and the inside of the head in basic image 6X is adopted as a feature of basic image 6X. More specifically, a pre-processing mask 640 defined by a ratio of the pixel value of a brain area of the head to a pixel value of a skin area of the head is generated as the feature of basic image 6X. Pre-processing mask 640 includes an area 641 corresponding to the brain, an area 642 corresponding to the skin, and an area 643 representing a margin portion in the outer periphery of the head.

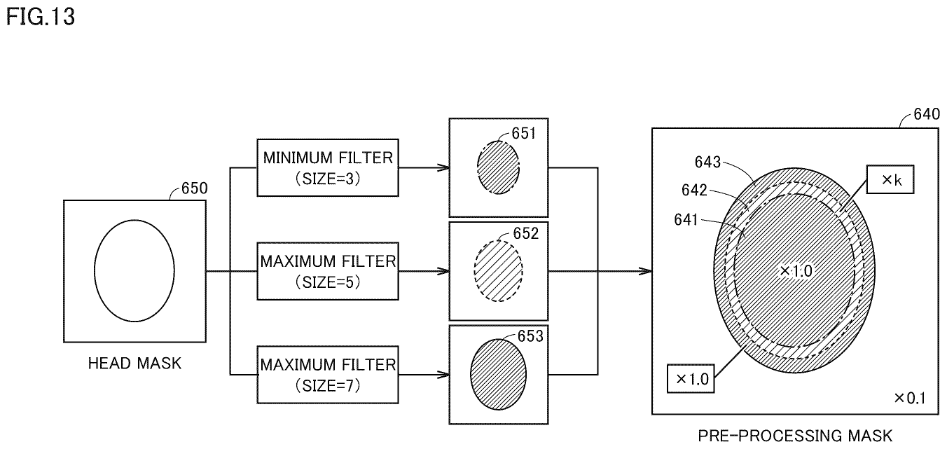

FIG. 13 is a diagram for more specifically illustrating the adjustment processing using pre-processing mask 640. A head mask 650 is a filter arranged at a position of the head in test image 6. Pre-processing mask 640 is configured by combination of head mask 650, a filter element 651 (a minimum filter having a size of 3) for area 641, a filter element 652 (a maximum filter having a size of 5) for area 642, and a filter element 653 (a maximum filter having a size of 7) for area 643 as being common in center of gravity. The inside of filter element 651 defines area 641. An inner side of filter element 652 on the outside of filter element 651 defines area 642. An inner side of filter element 653 on the outside of filter element 652 defines area 643.

Each of “x1.0”, “xk”, and “x0.1” in FIG. 13 represents a value by which a pixel value of target image 5 is to be multiplied in the adjustment processing.

More specifically, in FIG. 13, “x1.0” is given to area 641 and area 643, which means that a pixel value of a pixel located in these areas in target image 5 is to be multiplied by 1.0 in the adjustment processing.

In FIG. 13, “x0.1” is given to the area on the outside of area 643, which means that a pixel value of a pixel located in this area in target image 5 is to be multiplied by 0.1 in the adjustment processing.

In FIG. 13, “xk” is given to area 642, which means that a pixel value of a pixel located in this area in target image 5 is to be multiplied by k in the adjustment processing.

Value k is derived in accordance with an expression (3) below, the expression using statistical values of a plurality of basic images 6X.

$$k = x \times \frac{MED ( h )}{MED ( s )} \qquad ( 3 )$$

In the expression (3), a variable k is expressed as a product of a variable x and “MED(h)/MED(s)”. MED(h) represents a median value of pixel values of the brain area in the plurality of basic images 6X. MED(s) represents a median value of pixel values of the skin area in the plurality of basic images 6X. The area of the head as well as the skin area and the brain area in the head in each basic image 6X may be identified by the user or pattern recognition.

Variable x is a value defined by a type of a radiopharmaceutical agent to be used in measurement. In one implementation, a value of variable x is smaller in using fluorodeoxyglucose (FDG) than in using flutemetamol.

An upper limit and a lower limit may be set for variable k. An exemplary lower limit of variable k is 1.0.

When the lower limit of variable k is set to 1.0, in the adjustment processing where pre-processing mask 640 is used, the skin area is emphasized in target image 5 to generate test image 6. A degree of emphasis is dependent on relation between the statistical value of the pixel value in the skin area and the statistical value of the pixel value in the brain area in the plurality of basic images 6X. More specifically, when the median value of the pixel values in the skin area is equal to or smaller than x times as large as the median value of the pixel values in the brain area in the plurality of basic images 6X, the skin area is emphasized by multiplication of pixels in the skin area in target image 5 by k in the test image.

In the adjustment processing where pre-processing mask 640 is used, test image 6 is generated by multiplication of pixel values in an area on the outside of area 643 in target image 5 by 0.1. Thus, in the adjustment processing, noise in areas other than the head in target image 5 is canceled.

In the present embodiment described above, the test image is generated by pre-processing (adjustment processing) of the image to be applied to the machine learning model being performed on the target image. As the test image is then applied to the machine learning model, the intermediate image (an exemplary result of estimation) is generated. In the adjustment processing, the feature of the basic image used in the machine learning processing for training of the machine learning model is used.

The feature of the basic image corresponds to a domain where the basic image is obtained. For example, the position, the size, and/or an angle of the head in the basic image is/are dependent on positional relation between a bed (specifically, a location in the bed where the head is arranged) where the image of the head is taken and detector ring 2 in a facility where the basic image is obtained. In this case, an exemplary domain includes the positional relation in the facility. The feature of the basic image then corresponds to the positional relation in the facility where the basic image is obtained.

As the test image generated by the adjustment processing on the target image as above is applied to the machine learning model, an image subjected to processing for reduction of a domain gap between the target image and the basic image is applied to the machine learning model. The adjustment processing does not require a user to collect a large amount of data in an environment where the target image is collected. The data processing method according to the present disclosure can thus suppress burdens imposed on the user for reduction of the domain gap.

Training data to be used for training of machine learning model 8 is the pair of basic image 6X and intermediate image 7X which is a ground truth label for basic image 6X. In the training data, a type of data to be paired for use with the ground truth label is not limited to the image.

For example, basic image 6X may be data of a type other than an image, and may be data (basic data) obtained in a domain different from the domain where target image 5 is obtained. For example, waveform data of voice and sound or spectra is assumed as the basic data. In this case, an amount of noise is assumed as the feature of the basic data, and processing for adjusting the amount of noise, such as cancellation of noise, is assumed as the adjustment processing.

Even in such a case, in step S30 in the processing in FIG. 3, test image 6 is generated by the adjustment processing based on the feature of the basic data performed on target image 5. In step S40, test image 6 is applied to machine learning model 8 to generate intermediate image 7.

Timing of generation of the feature of the basic image is not restricted in any manner. The feature of the basic image may be generated in advance. In one implementation, data that defines the machine learning model and data that defines the feature of the basic image are stored in advance in the memory of controller 4. The feature of the basic image may be generated at the time of generation of the intermediate image in step S40.

### Aspects

Illustrative embodiments described above are understood by a person skilled in the art as specific examples of aspects below.

(Clause 1) A data processing method according to one aspect is a data processing method for generation of a test image, the data processing method being to be applied to a machine learning model subjected to machine learning processing using a basic image. The data processing method may include obtaining a target image in a domain different from a domain where the basic image is obtained and generating the test image by performing on the target image, adjustment processing based on a feature corresponding to the domain where the basic image is obtained.

According to the data processing method described in Clause 1, burdens imposed on a user for reduction of a domain gap can be suppressed.

(Clause 2) In the data processing method described in Clause 1, the target image may include a target object, the feature may be associated with a position of the target object in the basic image, and the adjustment processing may include adjusting the position of the target object in the test image.

According to the data processing method described in Clause 2, the domain gap in connection with the position of the target object can be reduced.

(Clause 3) In the data processing method described in Clause 1 or 2, the target image may include a target object, the feature may be associated with a shape of the target object in the basic image, and the adjustment processing may include adjusting the shape of the target object in the test image.

According to the data processing method described in Clause 3, the domain gap in connection with the shape of the target object can be reduced.

(Clause 4) In the data processing method described in any one of Clauses 1 to 3, the target image may include a target object, the feature may be associated with a posture of the target object in the basic image, and the adjustment processing may include adjusting the posture of the target object in the test image.

According to the data processing method described in Clause 4, the domain gap in connection with the posture of the target object can be reduced.

(Clause 5) In the data processing method described in any one of Clauses 1 to 4, the feature may be associated with contrast in the basic image, and the adjustment processing may include adjusting the contrast in the test image.

According to the data processing method described in Clause 5, the domain gap in connection with difference in contrast between the basic image and the target image can be reduced.

(Clause 6) In the data processing method described in any one of Clauses 1 to 5, the target image may include a target object, the feature may be associated with a ratio of a pixel value between an outer periphery and an inside of the target object, and the adjustment processing may include emphasis of the outer periphery in the target object.

According to the data processing method described in Clause 6, the domain gap in connection with the ratio of the pixel value between the outer periphery and the inside in the target object can be reduced.

(Clause 7) In the data processing method described in Clause 6, the target object may be an image of a head of a human, the outer periphery may correspond to a skin area of the head, and the inside may correspond to a brain area of the head.

According to the data processing method described in Clause 7, the domain gap in connection with the image of the head of a human can be reduced.

(Clause 8) In the data processing method described in Clause 6 or 7, the adjustment processing may use a filter, and the filter may be defined by a function of a statistical value of the pixel value of the outer periphery of at least one basic image and a statistical value of the pixel value of the inside of at least one basic image.

According to the data processing method described in Clause 8, the domain gap in connection with the image of the head of a human can more reliably be reduced.

(Clause 9) In the data processing method described in Clause 1, the adjustment processing may include canceling noise in an area other than the target object by using the filter.

According to the data processing method described in Clause 9, the test image in which noise has been canceled can be inputted to the machine learning model.

(Clause 10) In the data processing method described in any one of Clauses 1 to 9, the target image may be a positron emission tomography (PET) image or a single photon emission computed tomography (SPECT) image.

According to the data processing method described in Clause 10, in estimation using a PET image or a SPECT image, burdens imposed on the user for reduction of the domain gap can be suppressed.

(Clause 11) An attenuation coefficient image generation method according to one aspect may include generating an intermediate image including an image relating to a tissue area based on a test image generated according to the data processing method described in Clause 10 and generating an attenuation coefficient image based on the intermediate image and a known attenuation coefficient of the tissue area.

According to the attenuation coefficient image generation method described in Clause 11, burdens imposed on the user for reduction of the domain gap in generation of the attenuation coefficient image can be suppressed.

(Clause 12) An image generation method according to one aspect is an image generation method using a machine learning model. The machine learning model has been subjected to machine learning processing using basic data. The image generation method includes obtaining a target image obtained in a domain different from a domain where the basic data is obtained, generating a test image by performing on the target image, adjustment processing based on a feature corresponding to the domain where the basic data is obtained, and obtaining an output image by applying the test image to the machine learning model.

According to the image generation method described in Clause 12, burdens imposed on the user for reduction of the domain gap can be suppressed.

(Clause 13) A program according to one aspect, by being executed by at least one processor of a computer, may cause the computer to perform the data processing method described in any one of Clauses 1 to 10, the attenuation coefficient image generation method described in Clause 11, or the image generation method described in Clause 12.

According to the program described in Clause 13, burdens imposed on the user for reduction of the domain gap can be suppressed.

It should be understood that the embodiment disclosed herein is illustrative and non-restrictive in every respect. The scope of the present disclosure is defined by the terms of the claims rather than the description of the embodiment above and is intended to include any modifications within the scope and meaning equivalent to the terms of the claims. It is intended that each technique in the embodiment may be carried out alone or in combination with another technique in the embodiment as necessary, so long as the combination is possible.

What is claimed is:

1. A data processing method for generation of a test image, the data processing method being to be performed by a computer and applied to a machine learning model subjected to machine learning processing using a basic image, the data processing method comprising:
obtaining, by circuitry of the computer, a target image in a domain different from a domain where the basic image is obtained; and
generating, by the circuitry of the computer, the test image by performing on the target image, adjustment processing based on a feature corresponding to the domain where the basic image is obtained.

2. The data processing method according to claim 1, wherein
the target image includes a target object,
the feature is associated with a position of the target object in the basic image, and
the adjustment processing includes adjusting the position of the target object in the test image.

3. The data processing method according to claim 1, wherein
the target image includes a target object,
the feature is associated with a shape of the target object in the basic image, and
the adjustment processing includes adjusting the shape of the target object in the test image.

4. The data processing method according to claim 1, wherein
the target image includes a target object,
the feature is associated with a posture of the target object in the basic image, and
the adjustment processing includes adjusting the posture of the target object in the test image.

5. The data processing method according to claim 1, wherein
the feature is associated with contrast in the basic image, and
the adjustment processing includes adjusting the contrast in the test image.

6. The data processing method according to claim 1, wherein
the target image includes a target object,
the feature is associated with a ratio of a pixel value between an outer periphery and an inside of the target object, and
the adjustment processing includes emphasis of the outer periphery in the target object.

7. The data processing method according to claim 6, wherein
the target object is a head of a human,
the outer periphery corresponds to a skin area of the head, and
the inside corresponds to a brain area of the head.

8. The data processing method according to claim 6, wherein
the adjustment processing uses a filter, and
the filter is defined by a function of a statistical value of the pixel value of the outer periphery of at least one basic image and a statistical value of the pixel value of the inside of at least one basic image.

9. The data processing method according to claim 8, wherein
the adjustment processing includes canceling noise in an area other than the target object by using the filter.

10. The data processing method according to claim 1, wherein
the target image is a positron emission tomography (PET) image or a single photon emission computed tomography (SPECT) image.

11. An attenuation coefficient image generation method to be performed by a computer comprising:
generating, by circuitry of the computer, an intermediate image including an image relating to a tissue area based on a test image generated according to the data processing method according to claim 10; and
generating, by the circuitry of the computer, an attenuation coefficient image based on the intermediate image and a known attenuation coefficient of the tissue area.

12. An image generation method using a machine learning model, the image generation method being to be performed by a computer, the machine learning model having been subjected to machine learning processing using basic data, the image generation method comprising:
obtaining, by circuitry of the computer, a target image obtained in a domain different from a domain where the basic data is obtained;
generating, by the circuitry of the computer, a test image by performing on the target image, adjustment processing based on a feature corresponding to the domain where the basic data is obtained; and
obtaining, by the circuitry of the computer, an output image by applying the test image to the machine learning model.
